(19) United States
(12) Patent Application Publication
(10) Pub. No.: US 2025/0268978 A1
(43) Pub. Date: Aug. 28, 2025

(54) TOPICAL CREAM AND METHOD OF USE

(71) Applicant: WellSpring and Company LLC, Branson, MO (US)

(72) Inventors: HANNAH FORREST, Walnut Shade, MO (US); Hayden McNealy, Clever, MO (US)

(21) Appl. No.: 19/066,018

(22) Filed: Feb. 27, 2025

Related U.S. Application Data
(60) Provisional application No. 63/558,201, filed on Feb. 27, 2024.
(60) Provisional application No. 63/656,381, filed on Jun. 5, 2024.

(51) Int. Cl. A61K 38/12; A61K 9/16; A61K 31/4174; A61K 31/573; A61K 31/7048; A61K 47/44

(52) U.S. Cl. CPC A61K 38/12; A61K 9/16; A61K 31/4174; A61K 31/573; A61K 31/7048; A61K 47/44

(57) ABSTRACT

A topical cream composition is provided which comprises a mixture of a moisture barrier component, a steroid component, an antifungal component, and an antibiotic component. The combination of a moisture barrier, a steroid, an antifungal component, and an antibiotic component works together to optimally provide quick treatment and resolution for diaper dermatitis, also known as diaper rash, or other skin condition(s). The moisture barrier component provides a barrier between affected skin and diaper wetness; the steroid reduces swelling, itching, and/or other discomfort; the antifungal component treats any present fungal skin infection(s); and/or the antibiotic component treats any present bacterial skin infection(s). In one aspect, the topical cream comprises a composition of petroleum jelly, hydrocortisone cream, clotrimazole antifungal cream, and triple antibiotic ointment including bacitracin zinc, neomycin sulfate, and polymyxin B sulfate.

### CROSS-REFERENCE TO RELATED APPLICATIONS

This application claims priority pursuant to 35 U.S.C. § 119 (e) to co-pending U.S. Provisional Patent Application Ser. No. 63/558,201, filed Feb. 27, 2024, and to co-pending U.S. Provisional Patent Application Ser. No. 63/656,381, filed Jun. 5, 2024, the entire disclosures of which are incorporated herein by reference.

### FIELD OF THE INVENTION

The present invention relates generally to dermatological topicals. More specifically, the present invention is concerned with a topical cream for treating diaper rash and methods of use thereof.

### BACKGROUND OF THE INVENTION

Diaper dermatitis, also known as diaper rash, is a very common skin condition prevalent among newborns, infants, toddlers, and others who regularly wear diapers. Diaper rash forms as a red, inflamed, skin rash which typically starts around the buttocks and can spread to the thighs and genitals.

Diaper rash is often caused by prolonged skin contact with wet and/or soiled diapers, or in other words, by diapers not being changed often enough. Nonetheless, diaper rash can further be caused or worsened by chafing, bacterial or fungal infection, irritation from exposure to one or more new products or allergens, and/or other factors.

Traditionally, diaper rashes have been treated with creams containing zinc-oxide, which acts as moisture barrier between the affected skin and the wetness of a diaper. In addition to or in place of cream(s) containing zinc-oxide, leaving the affected area open to air for a period of time to fully dry is also a common treatment. Nevertheless, for severe outbreaks of diaper rash, traditional daily diaper creams and/or open-air drying are often insufficient to effectively treat the diaper rash. Accordingly, it would be beneficial to have a treatment which efficiently and effectively treats severe outbreaks of diaper rash.

There are several prescription creams and over the counter creams on the market to help treat diaper rash. However, currently available creams often require some trial and error before finding which one works for a given case of diaper rash. Additionally, use of such creams for prolonged periods of time can lead to further skin irritation or reaction. Accordingly, it would be beneficial to have one diaper cream which quickly treats diaper rash, no matter the underlying cause.

Heretofore, there has not been available a topical diaper cream and method of use with the advantages and features of the present invention.

### SUMMARY OF THE INVENTION

The present invention comprises a topical cream for treating diaper rash. In an embodiment, the present topical cream comprises a mixture of a moisture barrier component, a steroid component, an antifungal component, and an antibiotic component. In an exemplary embodiment, the combination of a moisture barrier, a steroid, an antifungal component, and an antibiotic component works together to optimally provide quick treatment and resolution for diaper rash. In an embodiment, the moisture barrier component provides a barrier between affected skin and diaper wetness; the steroid reduces swelling, itching, and/or other discomfort; the antifungal component treats any present fungal skin infection(s); and/or the antibiotic component treats any present bacterial skin infection(s). In embodiments, the present topical cream allows for quick and effective treatment of diaper rash, no matter the root cause of the diaper rash.

In an exemplary embodiment, the topical cream comprises a mixture of petroleum jelly, steroid cream, antifungal cream, and antibiotic ointment. In a preferred embodiment, the topical cream comprises a composition of petroleum jelly, hydrocortisone cream, clotrimazole antifungal cream, and triple antibiotic ointment. In alternative embodiments, triamcinolone acetonide steroid cream is used in place of or in addition to hydrocortisone cream.

In an exemplary embodiment, the present topical cream further includes an amount of aloe. In further embodiments, the topical cream includes one or more other additional anti-inflammatory component(s).

In an exemplary embodiment, the topical cream of the present invention includes an amount of lavender. In an exemplary embodiment, the topical cream of the present invention includes an amount of eucalyptus oil. In an embodiment, the topical cream includes a combination of aloe, lavender, and/or eucalyptus. In additional embodiments, one or more other essential oils are included within the topical cream of the present invention.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention and various features thereof.

### BRIEF DESCRIPTION OF THE DRAWINGS

Embodiments of the present invention are described in detail below with reference to the attached drawing figures, wherein:

FIG. 1 is a schematic diagram showing components of an embodiment of a topical cream composition embodying the present invention.

FIG. 2 is a flow chart showing a method of mixing an embodiment of a topical cream composition embodying the present invention.

FIG. 3 is a flow chart showing a process of treating diaper dermatitis with a topical cream of the present invention.

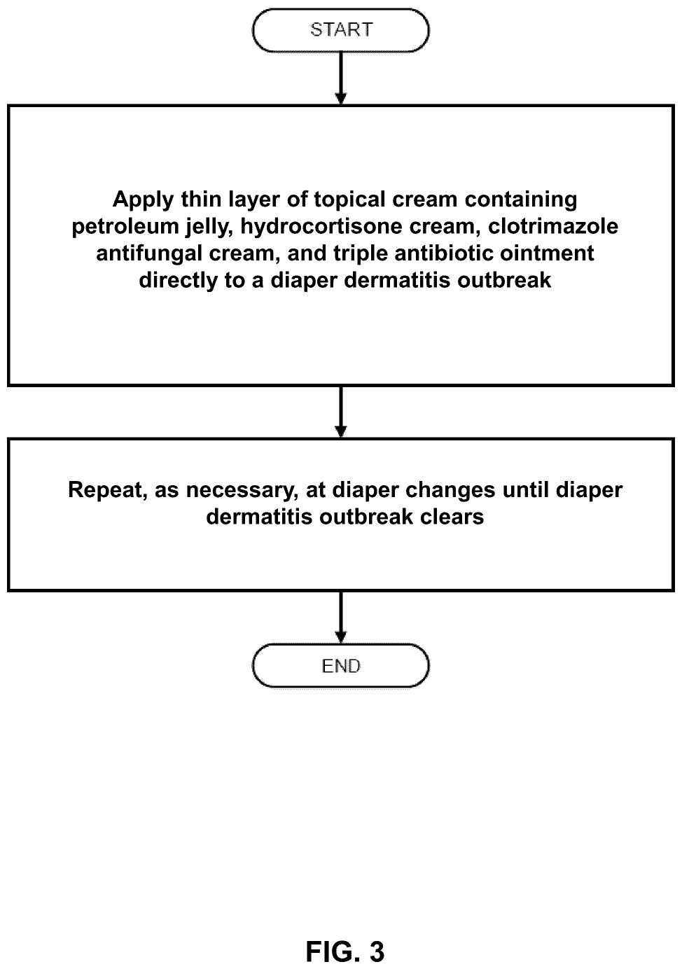

The drawing figures do not limit the present invention to the specific embodiments disclosed and described herein. The drawings are not necessarily to scale, emphasis instead being placed upon illustrating the principles of the invention.

### DETAILED DESCRIPTION OF THE PREFERRED EMBODIMENTS

As required, a detailed embodiment of the present invention is disclosed herein; however, it is to be understood that the disclosed embodiment is merely exemplary of the principles of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

The present invention comprises a topical cream composition and methods of composition and methods of use thereof. In an exemplary embodiment, the present topical cream composition is effective for and used in treatment of diaper dermatitis, also known as diaper rash. In other embodiments, the topical cream composition of the present invention is further effective for and used in treatment of other types of dermatitis, including but not limited to atopic dermatitis (also known as eczema), contact dermatitis, Seborrheic dermatitis, and/or nummular dermatitis, and/or any other type of skin condition or rash.

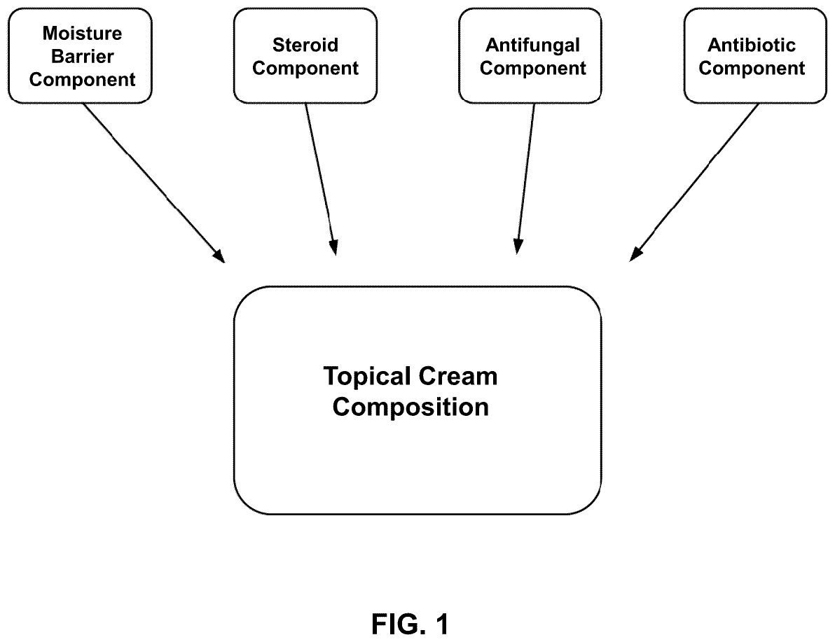

In an exemplary embodiment, the topical cream composition comprises a mixture including a moisture barrier component, a steroid component, an antifungal component, and an antibiotic component, as shown in FIG. 1. In an exemplary embodiment, the combination of a moisture barrier, a steroid, an antifungal component, and an antibiotic component works together to optimally provide quick treatment and resolution for diaper rash or another skin condition or rash.

In an embodiment, the moisture barrier component provides a barrier between affected skin and diaper wetness; the steroid reduces swelling, itching, and/or other discomfort; the antifungal component treats any present fungal skin infection(s); and/or the antibiotic component treats any present bacterial skin infection(s). In embodiments, the present topical cream allows for quick and effective treatment of diaper rash, no matter the root cause of the diaper rash. This is particularly advantageous for consumers because the root cause of diaper rash can often be uncertain to parents or other caregivers because of similarities in appearance of the rash regardless of the cause.

In an embodiment, the moisture barrier component comprises petroleum jelly. In an embodiment, the steroid component comprises a steroid cream. In an embodiment, the antifungal component comprises an antifungal cream. In an embodiment, the antibiotic component comprises an antibiotic ointment.

In an exemplary embodiment, the topical cream comprises a mixture of petroleum jelly, steroid cream, antifungal cream, and antibiotic ointment. In a preferred embodiment, the topical cream comprises a composition of petroleum jelly, hydrocortisone cream, clotrimazole antifungal cream, and triple antibiotic ointment. In alternative embodiments, triamcinolone acetonide steroid cream is used in place of or in addition to hydrocortisone cream.

In further embodiments, zinc-oxide, silicone, dimethicone, and/or another moisture barrier is utilized in place of or in addition to petroleum jelly.

In other embodiments, another topical corticosteroid and/or other steroid other than hydrocortisone or triamcinolone acetonide is utilized in place of or in addition to hydrocortisone and/or triamcinolone acetonide.

In further yet embodiments, another antifungal component other than clotrimazole, such as but not limited to miconazole, ketoconazole, and/or nystatin is utilized in place of or in addition to clotrimazole.

In additional embodiments, antibiotic component(s) other than a combination of bacitracin zinc, neomycin sulfate, and polymyxin B sulfate is utilized, including but not limited to bacitracin zinc alone; neomycin sulfate alone; polymyxin B sulfate alone; any combination of bacitracin zinc, neomycin sulfate, and polymyxin B sulfate; mupirocin; bacitracin; and/or any other antibiotic component(s).

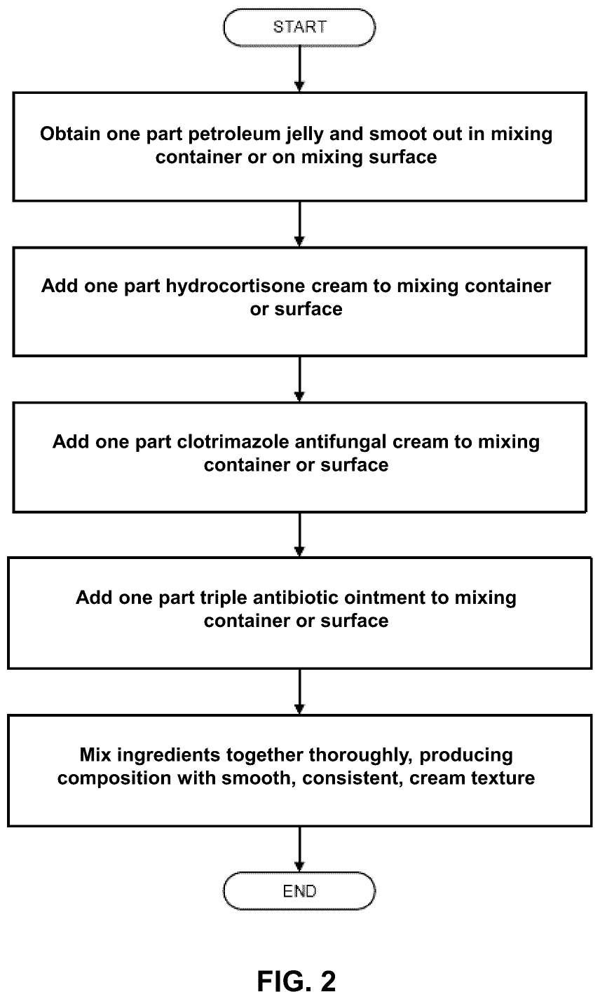

FIG. 2 shows an exemplary embodiment of a method of making a topical cream composition of the present invention. In an exemplary embodiment, to produce a topical cream of the present invention, equal parts by weight of petroleum jelly, hydrocortisone cream, clotrimazole antifungal cream, and triple antibiotic ointment are combined and mixed together. In a further embodiment, about equal parts of petroleum jelly, hydrocortisone cream, clotrimazole antifungal cream, and triple antibiotic ointment are combined and mixed together. In exemplary embodiments, the mixed composition forms a cream with a soft, smooth, and consistent texture. In an exemplary embodiment, the components of the present topical cream are mixed together at room temperature. In other embodiments, the components are mixed at colder or warmer temperatures than room temperature.

In an exemplary embodiment of a method of making a topical cream of the present invention, one part, by weight, of petroleum jelly is spread and smoothed out in a mixing container. Then, one part, by weight, of each of the other components is added into the mixing container, one by one, and mixed in with a spatula or similar mixing tool or device. In embodiments, the order of mixing in hydrocortisone cream, clotrimazole antifungal cream, and triple antibiotic ointment into the composition mixture can be varied or done in any order. Alternatively, in embodiments, equal parts, by weight, of all of the petroleum jelly, hydrocortisone cream, clotrimazole antifungal cream, and triple antibiotic ointment are combined at the same time and mixed together with a spatula or similar mixing tool or device. In some embodiments, a hand mixer or commercial mixer is utilized to achieve an even smoother consistency of the topical cream composition.

In alternative embodiments, a different moisture barrier component than petroleum jelly, such as but not limited to zinc-oxide, silicone, dimethicone, and/or another moisture barrier, is used but combined in the same manner discussed above relating to petroleum jelly.

In other embodiments, a different steroid component than hydrocortisone, such as but not limited to triamcinolone acetonide, another topical corticosteroid, and/or other steroid, is used but combined in the same manner discussed above relating to hydrocortisone cream.

In further embodiments, a different antifungal component than clotrimazole, such as but not limited to miconazole, ketoconazole, and/or nystatin, is used but combined in the same manner discussed above relating to clotrimazole antifungal cream.

In embodiments, a different antibiotic component than triple antibiotic cream, such as but not limited to bacitracin zinc alone; neomycin sulfate alone; polymyxin B sulfate alone; any subset combination of bacitracin zinc, neomycin sulfate, and polymyxin B sulfate; mupirocin; bacitracin; and/or any other antibiotic component(s), is used but combined in the same manner discussed above relating to triple antibiotic cream.

In embodiments, the moisture barrier component makes up 10%, 15%, 20%, 25%, 30%, 35%, 40%, 45%, 50%, 10%-50%, 15%-35%, about 10%, about 15%, about 20%, about 25%, about 30%, about 35%, about 40%, about 45%, about 50%, about 10%-50%, about 15%-35%, by weight, of the total topical cream composition.

In an exemplary embodiment of the present invention, the pre-mixture or feedstock hydrocortisone cream component mixed into the topical cream composition comprises a cream containing 1%, by weight, hydrocortisone. In further embodiments, the pre-mixture or feedstock hydrocortisone cream component contains 0.25%, 0.5%, 0.75%, 1.25%, 1.5%, 1.75%, 2%, 2.25%, 2.5%, 2.75%, 3%, 3.25%, 3.5%, 3.75%, 4%, 4.25%, 4.5%, 4.75%, 5%, 5.5%, 6%, 6.5%, 7%, 7.5%, 8%, about 0.25%, about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.5%, about 6%, about 6.5%, about 7%, about 7.5%, or about 8%, by weight, hydrocortisone. In embodiments, topical cream of the present invention includes 0.1%, 0.2%, 0.25%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.75%, 3%, 3.25%, 3.5%, 3.75%, 4%, about 0.1%, about 0.2%, about 0.25%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.75%, about 3%, about 3.25%, about 3.5%, about 3.75%, about 4%, 0.1%-4%, 0.25%-2.5%, 0.25%-1%, about 0.1%-4%, about 0.25%-2.5%, or about 0.25%-1%, by weight, hydrocortisone of the total topical cream composition. In embodiments, the topical cream composition of the present invention comprises 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, 2 mg-12 mg, 2.5 mg-10 mg, about 2 mg-about 12 mg, or 2.5 mg-10 mg of hydrocortisone per gram of the total topical cream composition. Nevertheless, other amounts of steroid by weight are utilized in other embodiments of the present invention.

In an alternative embodiment of the present invention, a pre-mixture or feedstock triamcinolone acetonide cream component mixed into the topical cream composition comprises a cream containing 0.025%, 0.05%, 0.075%, 0.1%, 0.2%, 0.3%, 0.4%, 0.5%, 0.6%, 0.7%, 0.8%, 0.9%, 1%, about 0.025%, about 0.05%, about 0.075%, about 0.1%, about 0.2%, about 0.3%, about 0.4%, about 0.5%, about 0.6%, about 0.7%, about 0.8%, about 0.9%, or about 1%, by weight, triamcinolone acetonide of a total topical cream composition.

In an embodiment, the pre-mixture or feedstock clotrimazole cream component mixed into the topical cream composition comprises a cream containing 1% by weight clotrimazole. In further embodiments, the pre-mixture or feedstock clotrimazole cream component contains 0.25%, 0.5%, 0.75%, 1.25%, 1.5%, 1.75%, 2%, 2.25%, 2.5%, 2.75%, 3%, 3.25%, 3.5%, 3.75%, 4%, 4.25%, 4.5%, 4.75%, 5%, 5.5%, 6%, 6.5%, 7%, 7.5%, 8%, about 0.25%, about 0.5%, about 0.75%, about 1%, about 1.25%, about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75%, about 5%, about 5.5%, about 6%, about 6.5%, about 7%, about 7.5%, or about 8%, by weight, clotrimazole. In embodiments, topical cream of the present invention includes 0.1%, 0.2%, 0.25%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.75%, 3%, 3.25%, 3.5%, 3.75%, 4%, about 0.1%, about 0.2%, about 0.25%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.75%, about 3%, about 3.25%, about 3.5%, about 3.75%, about 4%, 0.1%-4%, 0.25%-2%, 0.25%-1%, about 0.1%-4%, about 0.25%-2%, or about 0.25%-1%, by weight, clotrimazole of the total topical cream composition. In embodiments, the topical cream composition of the present invention comprises 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, 2 mg-12 mg, 2.5 mg-10 mg, about 2 mg-about 12 mg, or 2.5 mg-10 mg of clotrimazole per gram of the total topical cream composition. Nevertheless, other amounts of antifungal by weight are utilized in other embodiments of the present invention.

In an embodiment, the pre-mixture or feedstock triple antibiotic ointment component mixed into the topical cream composition comprises 400 units of bacitracin zinc, 3.5 mg of neomycin sulfate, and 5,000 units of polymyxin B sulfate per gram of the pre-mixture or feedstock ointment.

In embodiments, the topical cream comprises 0.5 mg, 0.6 mg, 0.7 mg, 0.8 mg, 0.875 mg, 0.9 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, about 0.5 mg, about 0.6 mg, about 0.7 mg, about 0.8 mg, about 0.875 mg, about 0.9 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, 0.5 mg-5 mg, 0.875 mg-3.5 mg, about 0.5 mg-about 5 mg, or about 0.875 mg-about 3.5 mg of neomycin sulfate per gram of the total topical cream composition. In embodiments, the topical cream comprises 0.05%-1%, 0.0875%-0.35%, about 0.05%-about 1%, or about 0.0875%-about 0.35% neomycin sulfate, by weight, of the total topical cream composition.

In embodiments, the topical cream comprises 25 units, 50 units, 75 units, 100 units, 125 units, 150 units, 175 units, 200 units, 225 units, 250 units, 275 units, 300 units, 325 units, 350 units, 375 units, 425 units, 450 units, 475 units, 500 units, 525 units, 550 units, 575 units, 600 units, 625 units, 650 units, 675 units, 700 units, 725 units, 750 units, 800 units, 850 units, 900 units, 950 units, 1000 units, about 25 units, about 50 units, about 75 units, about 100 units, about 125 units, about 150 units, about 175 units, about 200 units, about 225 units, about 250 units, about 275 units, about 300 units, about 325 units, about 350 units, about 375 units, about 400 units, about 425 units, about 450 units, about 475 units, about 500 units, about 525 units, about 550 units, about 575 units, about 600 units, about 625 units, about 650 units, about 675 units, about 700 units, about 725 units, about 750 units, about 800 units, about 850 units, about 900 units, about 950 units, about 1000 units, 25 units-1000 units, 100 units-500 units, about 25 units-about 1000 units, or about 100 units-about 500 units of bacitracin zinc per gram of the total topical cream composition.

In embodiments, the topical cream comprises 800 units, 900 units, 1,000 units, 1,250 units, 1,500 units, 1,750 units, 2,000 units, 2,250 units, 2,500 units, 2,750 units, 3,000 units, 3,250 units, 3,500 units, 3,750 units, 4,000 units, 4,250 units, 4,500 units, 4,750 units, 5,250 units, 5,500 units, 5,750 units, 6,000 units, 6,500 units, 7,000 units, 7,500 units, 8,000 units, 8,500 units, 9,000 units, 9,500 units, 10,000 units, 11,000 units, 12,000 units, about 800 units, about 900 units, about 1,000 units, about 1,250 units, about 1,500 units, about 1,750 units, about 2,000 units, about 2,250 units, about 2,500 units, about 2,750 units, about 3,000 units, about 3,250 units, about 3,500 units, about 3,750 units, about 4,000 units, about 4,250 units, about 4,500 units, about 4,750 units, about 5,000 units, about 5,250 units, about 5,500 units, about 5,750 units, about 6,000 units, about 6,500 units, about 7,000 units, about 7,500 units, about 8,000 units, about 8,500 units, about 9,000 units, about 9,500 units, about 10,000 units, about 11,000 units, about 12,000 units, 800 units-12,000 units, 1,250 units-10,000 units, about 800 units-about 12,000 units, or about 1,250 units-about 10,000 units of polymyxin B sulfate per gram of the total topical cream composition. Nevertheless, other antibiotics and/or varied amounts of antibiotics are utilized in other embodiments.

In an exemplary embodiment, the present topical cream further includes an amount of aloe. In an embodiment, an aloe component mixed into the topical cream composition is aloe vera. In further embodiments, the topical cream includes one or more other additional anti-inflammatory component(s).

In an exemplary embodiment, the topical cream of the present invention includes an amount of lavender. In a further exemplary embodiment, the topical cream of the present invention includes an amount of eucalyptus oil. In another embodiment of the present invention, the topical cream includes lavender and eucalyptus. In an embodiment, the topical cream includes some combination of aloe, lavender, and/or eucalyptus. In additional embodiments, one or more other essential oils are included within the topical cream of the present invention.

In an exemplary embodiment, the present topical cream is applied to an area of skin affected with diaper rash during every diaper change over the course of one, two, or three days. In another embodiment, the present topical cream is applied to an area of skin affected with diaper rash every other diaper change over the course of one, two, or three days. In other embodiments, the present topical cream is applied to an area of skin affected with diaper rash once a day, twice a day, three times a day, four times a day, or as needed. In exemplary embodiments, the present topical cream is configured to clear up diaper rash within one day, two days, three days, or four days. In an embodiment, a thin layer of the topical cream of the present invention is applied directly to a diaper dermatitis outbreak and repeated as needed until the outbreak clears up.

Certain terminology is used in the description for convenience in reference only and will not be limiting. For example, up, down, front, back, right, and left refer to the invention as orientated in the view being referred to. The words “inwardly” and “outwardly” refer to directions toward and away from, respectively, the geometric center of the aspect being described and designated parts thereof. Forwardly and rearwardly are generally in reference to the direction of travel, if appropriate. Additionally, anatomical terms are given their usual meanings. For example, proximal means closer to the trunk of the body, and distal means further from the trunk of the body. Said terminology shall include the words specifically mentioned, derivatives thereof, and words of similar meaning.

As used in this specification and the appended claims, the singular forms “a”, “an”, and “the” include plural references unless the context clearly dictates otherwise. Thus, for example, a reference to “a method” includes one or more methods, elements, and/or steps of the type described herein and/or which will become apparent to those persons skilled in the art upon reading this disclosure and so forth.

As used in this specification and the appended claims, the use of the term “about” means a range of values including and within 15% above and below the named value, except for nominal temperature. For example, the phrase “about 3 mM” means within 15% of 3 mM, or 2.55-3.45, inclusive. Likewise, the phrase “about 3 millimeters (mm)” means 2.55 mm-3.45 mm, inclusive. When temperature is used to denote change, the term “about” means a range of values including and within 15% above and below the named value. For example, “about 5° C.,” when used to denote a change such as in “a thermal resolution of better than 5° C. across 3 mm,” means within 15% of 5° C., or 4.25° C.-5.75° C. When referring to nominal temperature, such as “about −50° C. to about +50° C.,” the term “about” means ±5° C. Thus, for example, the phrase “about 37° C.” means 32° C.-42° C.

Unless defined otherwise, all technical and scientific terms used herein have the same meaning as commonly understood by one of ordinary skill in the art to which this invention belongs. Although any systems, elements, methods and materials similar or equivalent to those described herein can be used in the practice or testing of the present invention, the preferred systems, elements, and methods and materials are now described. All publications mentioned herein are incorporated herein by reference to describe in their entirety.

“Substantially” means to be more-or-less conforming to the particular dimension, range, shape, concept, or other aspect modified by the term, such that a feature or component need not conform exactly. For example, a “substantially cylindrical” object means that the object resembles a cylinder but may have one or more deviations from a true cylinder. “Comprising,” “including,” and “having” (and conjugations thereof) are used interchangeably to mean including but not necessarily limited to, and are open-ended terms not intended to exclude additional, unrecited elements or method steps.

Changes may be made in the above methods, devices and structures without departing from the scope hereof. Many different arrangements of the various components depicted, as well as components not shown, are possible without departing from the spirit and scope of the present invention. Embodiments of the present invention have been described with the intent to be illustrative and exemplary of the invention, rather than restrictive or limiting of the scope thereof. Alternative embodiments will become apparent to those skilled in the art that do not depart from its scope. Specific structural and functional details disclosed herein are not to be interpreted as limiting, but merely as a representative basis for teaching one of skill in the art to employ the present invention in any appropriately detailed structure. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described.

It is also to be understood that the following claims are intended to cover all of the generic and specific features of the invention herein described, and all statements of the scope of the invention which, as a matter of language, might be said to fall therebetween.

1. A topical cream composition comprising:
a moisture barrier component;
a steroid component;
an antifungal component; and
an antibiotic component;
wherein said moisture barrier component, said steroid component, said antifungal component, and said antibiotic component are mixed together into a smooth, consistent, cream texture.

2. The topical cream composition according to claim 1, wherein:
said moisture barrier component comprises petroleum jelly.

3. The topical cream composition according to claim 1, wherein:
said moisture barrier component makes up about 15% to about 35%, by weight, of said topical cream composition.

4. The topical cream composition according to claim 1, wherein:
said steroid component comprises hydrocortisone cream.

5. The topical cream composition according to claim 4, wherein:
said topical cream composition comprises about 0.25% to about 2.5% hydrocortisone, by weight, of said topical cream composition.

6. The topical cream composition according to claim 1, wherein:
said antifungal component comprises clotrimazole antifungal cream.

7. The topical cream composition according to claim 6, wherein:
said topical cream composition comprises about 0.25% to about 2% clotrimazole, by weight, of said topical cream composition.

8. The topical cream composition according to claim 1, wherein:
said antibiotic component comprises triple antibiotic ointment.

9. The topical cream composition according to claim 8, wherein:
said triple antibiotic ointment comprises bacitracin zinc, neomycin sulfate, and polymyxin B sulfate.

10. The topical cream composition according to claim 9, wherein:
said topical cream composition comprises about 0.875 mg to about 3.5 mg of neomycin sulfate per gram of said topical cream composition.

11. The topical cream composition according to claim 9, wherein:
said topical cream composition comprises about 100 units to about 500 units of bacitracin zinc per gram of said topical cream composition.

12. The topical cream composition according to claim 9, wherein:
said topical cream composition comprises about 1,250 units to about 10,000 units of polymyxin B sulfate per gram of said topical cream composition.

13. The topical cream composition according to claim 1, wherein:
said steroid component comprises triamcinolone acetonide cream.

14. The topical cream composition according to claim 13, wherein:
said topical cream composition comprises about 0.025% to about 0.5% triamcinolone, by weight, of said topical cream composition.

15. The topical cream composition according to claim 1, wherein:
said topical cream composition is configured to treat diaper dermatitis.

16. A method of making a topical cream composition, the method comprising:
providing a moisture barrier component, a steroid component, an antifungal component, and an antibiotic component; and
mixing said moisture barrier component, said steroid component, said antifungal component, and said antibiotic component together into a topical cream composition with a smooth, consistent, cream texture.

17. The method of claim 16, wherein:
said moisture barrier component comprises petroleum jelly;
said steroid component comprises hydrocortisone cream;
said antifungal component comprises clotrimazole antifungal cream; and
said antibiotic component comprises triple antibiotic ointment including bacitracin zinc, neomycin sulfate, and polymyxin B sulfate.

18. The method of claim 17, wherein said topical cream composition comprises:
about 15% to about 35% petroleum jelly, by weight, of said topical cream composition;
about 0.25% to about 2.5% hydrocortisone, by weight, of said topical cream composition;
about 0.25% to about 2% clotrimazole, by weight, of said topical cream composition;
about 0.875 mg to about 3.5 mg of neomycin sulfate per gram of said topical cream composition;
about 100 units to about 500 units of bacitracin zinc per gram of said topical cream composition; and
about 1,250 units to about 10,000 units of polymyxin B sulfate per gram of said topical cream composition.

19. A method of treating diaper dermatitis comprising:
providing a topical cream composition comprising a mixture of a moisture barrier component, a steroid component, an antifungal component, and an antibiotic component;
applying a thin layer of said topical cream composition over a diaper dermatitis outbreak; and
repeating as necessary until said diaper dermatitis outbreak clears.

20. The method of claim 19, wherein:
said moisture barrier component comprises petroleum jelly;
said steroid component comprises hydrocortisone cream;
said antifungal component comprises clotrimazole antifungal cream; and
said antibiotic component comprises triple antibiotic ointment including bacitracin zinc, neomycin sulfate, and polymyxin B sulfate.
